(19) United States
(12) Patent Application Publication
(10) Pub. No.: US 2010/0063561 A1
(43) Pub. Date: Mar. 11, 2010

(54) IMPLANTABLE CARDIAC STIMULATION DEVICES WITH SAFE-MODE OPERATION

(75) Inventors: Laurence S. Sloman, West Hollywood, CA (US); Jeffery D. Snell, Chatsworth, CA (US)

Correspondence Address: PACESETTER, INC., 15900 VALLEY VIEW COURT, SYLMAR, CA, 91392-9221, US

(73) Assignee: PACESETTER, INC., Sylmar, CA (US)

(21) Appl. No.: 12/205,662

(22) Filed: Sep. 5, 2008

(51) Int. Cl. A61N 1/37

(52) U.S. Cl. 607/27

(57) ABSTRACT

A plurality of electrodes are implanted in, on or near the patient's heart and initially configured to define first circuits or vectors enabled for at least one of sensing and stimulating and second circuits or vectors which are idle for at least one of sensing and stimulating. Selected first circuits or second circuits are tested for fault indications related to one or both of sensing and stimulating and a status record is updated to indicate corresponding sensing fault indications and stimulating fault indications. If a sensing fault is found in one of the first circuits, the first circuit is redefined when enabled for sensing to include at least one electrode of a second circuit that does not have a record of a sensing fault indication. Likewise, if a stimulating fault is found in one of the first circuits, the first circuit is redefined when enabled for stimulating to include at least one electrode of a second circuit that does not have a record of a stimulating fault indication.

### FIELD OF THE INVENTION

The invention relates to the field of implantable medical devices and to systems and methods of providing back-up or safe mode operation in case of lead circuit compromise.

### BACKGROUND OF THE INVENTION

A variety of implantable medical devices are known to automatically monitor a patient's physiologic condition and to selectively provide therapy when indicated. Implantable pacemakers and/or cardioverter defibrillators (ICDs) are implantable medical devices which are configured to monitor a patient's cardiac activity and selectively provide therapy for a variety of cardiac arrhythmias. Implantable pacemakers and/or ICDs typically include a stimulation pulse generator which generates therapeutic stimulation for delivery to patient tissue and a microprocessor-based controller which regulates the delivery of that therapy. The stimulation pulse generator and controller circuitry is generally encased within a biocompatible can or housing along with a battery to power the device.

Implantable pacemakers and/or ICDs typically also include one or more implantable patient leads with associated electrodes. The patient leads include insulated conductors connected at one end to a corresponding electrode and at the other with the stimulation pulse generator and controller in the can or housing. The patient leads are configured for transvenous catheterization to place the electrodes into contact with the patient's cardiac tissue. The leads also typically include some sort of structure or fixation method to secure the leads in place once implanted.

The leads provide the ability to conduct the therapeutic stimulation remotely from the stimulation pulse generator and controller in the can or housing which is generally positioned some distance from the patient's heart for delivery to target regions of the patient's heart. The leads also provide the function of conducting physiologically-based electrical signals arising from cardiac depolarizations, as well as other sources to the remotely located housing and controller. Thus, the implantable patient leads are frequently employed in a time multiplexed manner to both convey stimulation signals to the patient's cardiac tissue, as well as to conduct activity signals from the patient's cardiac tissue.

As the patient leads are configured for intravenous placement, they are correspondingly made relatively thin to avoid occlusion of the venous passages in which they reside. However, the thinness of the patient leads, as well as the nature of fixing these leads on living moving tissue, presents certain difficulties. More particularly, the patient leads include relatively thin insulated conductors and are also made to be at least partially flexible to accommodate passage through curved vasculature, as well as to accommodate internal movement within the veins and more particularly adjacent the beating heart. The thin nature of the insulated conductors, as well as the constant exposure to vibration and physical movement due to the beating heart muscle as well as other patient movement, can cause the individual insulated conductors within the patient leads to fracture.

While the conductive portions of the patient leads are constrained and encased by insulative material, a fracture in the conductive material tends to result in at best a problematic conductive path between the distal electrodes and the implantable device housing, resulting in intermittent conductivity therebetween or in a worst case, a complete open circuit. Fractures in the lead conductors may develop over time, and the continuity exhibited by a fractured or fracturing conductor can progressively deteriorate over time and is thus not always sudden and catastrophic. For example, an incipient lead fracture may manifest itself as a deterioration in the ability of the patient lead to properly conduct relatively low amplitude physiologic signals, yet the lead may maintain the ability to reliably deliver the relatively higher amplitude therapeutic stimulations to the patient tissue.

Another difficulty occurs when the patient lead dislodges from the desired implanted location. This can arise simply due to the repeated mechanical stresses induced by the beating heart and other muscle and structural movement arising from the patient's normal activity. Lead dislodgment can also be caused by nervousness or irritation on the part of the patient resulting in “picking” at the implanted lead. Lead dislodgement is particularly troublesome as not only are the corresponding electrodes removed from the desired implanted location, thereby compromising their ability to sense the cardiac activity of interest, but the ability to deliver therapy to the desired target location is also limited. A dislodged lead will also, assuming the conductivity of the lead and electrodes remains intact, place these electrodes in a new, possibly troublesome location. More particularly, the electrodes will still sense physiologic activity, however, not from the intended location. This changed sensing may confound the ability of the device to accurately sense the true physiologic activity occurring. A further concern is that a dislodged lead can deliver therapeutic stimulation to a location other than that intended, and this can result in unwanted and even dangerous stimulation to the patient.

### SUMMARY OF THE INVENTION

From the foregoing, it will be appreciated that there is a need and desire for implantable medical device systems and methods of operating these systems to better accommodate unexpected lead problems. It would be desirable for such new systems and methods of operation to provide back-up or redundancies to maintain the ability to sense cardiac activity in case of lead damage in a primary or initial circuit. There is also a need and desire for a safe mode type of operation to avoid delivery of stimulation to regions other than those intended, e.g., in cases of lead dislodgement. There is also a desire for new systems and methods which can at least partially automatically operate to provide an intervention in the interval between a component problem and the attention of a physician or other clinician to address the underlying cause, such as by replacement of a faulty lead or reattachment of a dislodged lead.

These needs are satisfied by the invention which, in one aspect, relates to a method of monitoring a patient and providing therapy via an implantable medical device. The method includes implanting a plurality of electrodes in, on or near the patient's heart, and initially configuring the plurality of electrodes to define first circuits enabled for at least one of sensing and stimulating and second circuits which are idle for at least one of sensing and stimulating. The method also includes testing selected first circuits or second circuits for fault indications related to one or both of sensing and stimulating and updating a status record to indicate corresponding sensing fault indications and stimulating fault indications. If a sensing fault is found in one of the first circuits, the method further includes redefining the first circuit when enabled for sensing to include at least one electrode of a second circuit that does not have a record of a sensing fault indication. Likewise, if a stimulating fault is found in one of the first circuits, the method further includes redefining the first circuit when enabled for stimulating to include at least one electrode of a second circuit that does not have a record of a stimulating fault indication.

Another aspect of the invention relates to an implantable cardiac stimulation device that includes an implantable stimulation generator, a plurality of implantable electrodes and a controller in communication with the stimulation generator and the plurality of electrodes. The controller activates selected ones of the plurality of electrodes to form an active circuit for at least one of sensing physiologic activity and delivering therapy from the stimulation generator to patient tissue, and idles selected other ones of the plurality of electrodes to form one or more idle circuits as alternatives to the active circuit for possible later activation. The controller also evaluates the active circuit for problem indications and, upon detecting a problem, designates the active circuit as unavailable and activates at least one of the idled circuits. The device further includes memory in communication with the controller and configured to store a status record of the active, idle, or unavailable state of the plurality of circuits.

These and other objects and advantages of the invention will become more apparent from the following description taken in conjunction with the accompanying drawings.

### BRIEF DESCRIPTION OF THE DRAWINGS

FIG. 1 is a simplified diagram illustrating an implantable stimulation device in electrical communication with at least three leads implanted into a patient's heart for delivering multi-chamber pacing stimulation and defibrillation or cardioversion therapy;

FIG. 2 is a functional block diagram of a multi-chamber implantable stimulation device illustrating the basic elements of a stimulation device which can provide cardioversion, defibrillation and pacing stimulation in four chambers of the heart;

FIG. 3 is a flow-chart of a system and method of providing a safe or back-up mode of operation in an implantable medical device in case of circuit problems;

FIG. 4A is a block diagram of an initial status table indicating active or enabled circuits, available circuits that are redundant or idle, and faulty or unavailable circuits;

FIG. 4B is a block diagram of subsequent initial status table indicating active or enabled circuits, available circuits that are redundant or idle, and faulty or unavailable circuits updated with respect to the table of FIG. 4A following a fault; and

FIG. 4C is a block diagram of a further embodiment of a status table indicating active or enabled circuits, available circuits that are redundant or idle, and faulty or unavailable circuits.

### DETAILED DESCRIPTION OF THE PREFERRED EMBODIMENTS

Reference will now be made to the drawings wherein like numerals refer to like parts throughout. The following description is of the best mode presently contemplated for practicing the invention. This description is not to be taken in a limiting sense but is made merely for the purpose of describing the general principles of the invention. The scope of the invention should be ascertained with reference to the issued claims. In the description of the invention that follows, like numerals or reference designators will be used to refer to like parts or elements throughout.

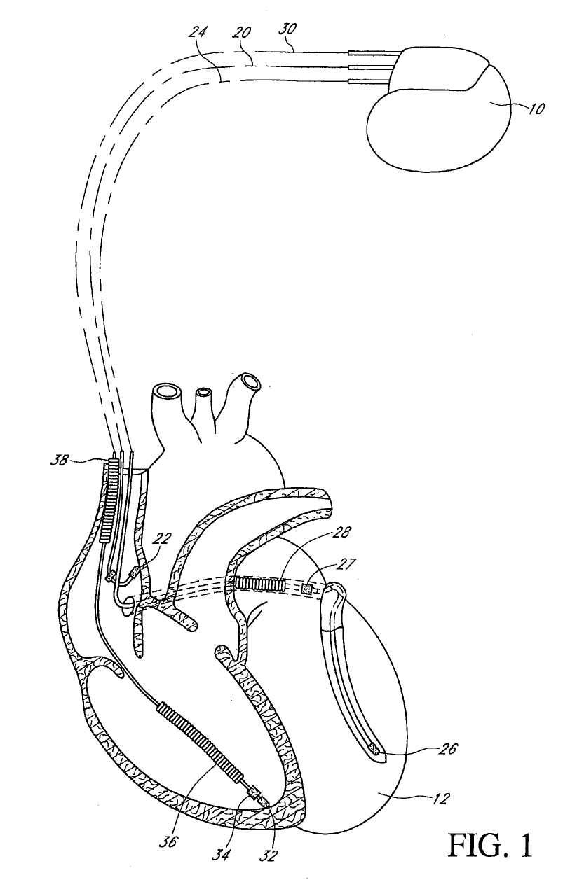

In one embodiment, as shown in FIG. 1, an implantable cardiac stimulation device 10 is in electrical communication with a patient's heart 12 by way of three leads, 20, 24 and 30, suitable for delivering multi-chamber stimulation and high voltage shock therapy. To sense atrial cardiac signals and to provide right atrial chamber stimulation therapy, the stimulation device 10 is coupled to an implantable right atrial lead 20 having at least an atrial tip electrode 22, which typically is implanted in the patient's right atrial appendage.

To sense left atrial and ventricular cardiac signals and to provide left chamber pacing therapy, the stimulation device 10 is coupled to a “coronary sinus” lead 24 designed for placement in the “coronary sinus region” via the coronary sinus ostium (OS) for positioning a distal electrode 26 adjacent to the left ventricle and/or additional electrode(s) 27, 28 adjacent to the left atrium. As used herein, the phrase “coronary sinus region” refers to the vasculature of the left ventricle, including any portion of the coronary sinus, great cardiac vein, left marginal vein, left posterior ventricular vein, middle cardiac vein, and/or small cardiac vein or any other cardiac vein accessible by the coronary sinus.

Accordingly, an exemplary coronary sinus lead 24 is designed to receive atrial and ventricular cardiac signals and to deliver left ventricular pacing therapy using at least a left ventricular tip electrode 26, left atrial pacing therapy using at least a left atrial ring electrode 27, and high voltage shocking therapy using at least a left atrial coil electrode 28.

The stimulation device 10 is also shown in electrical communication with the patient's heart 12 by way of an implantable right ventricular lead 30 having, in this embodiment, a right ventricular tip electrode 32, a right ventricular ring electrode 34, a right ventricular (RV) coil electrode 36, and a superior vena cava (SVC) coil electrode 38. Typically, the right ventricular lead 30 is transvenously inserted into the heart 12 so as to place the right ventricular tip electrode 32 in the right ventricular apex so that the RV coil electrode will be positioned in the right ventricle and the SVC coil electrode 38 will be positioned in the superior vena cava. Accordingly, the right ventricular lead 30 is capable of receiving cardiac signals, and delivering stimulation in the form of pacing, cardioversion, or defibrillation therapy to the right ventricle.

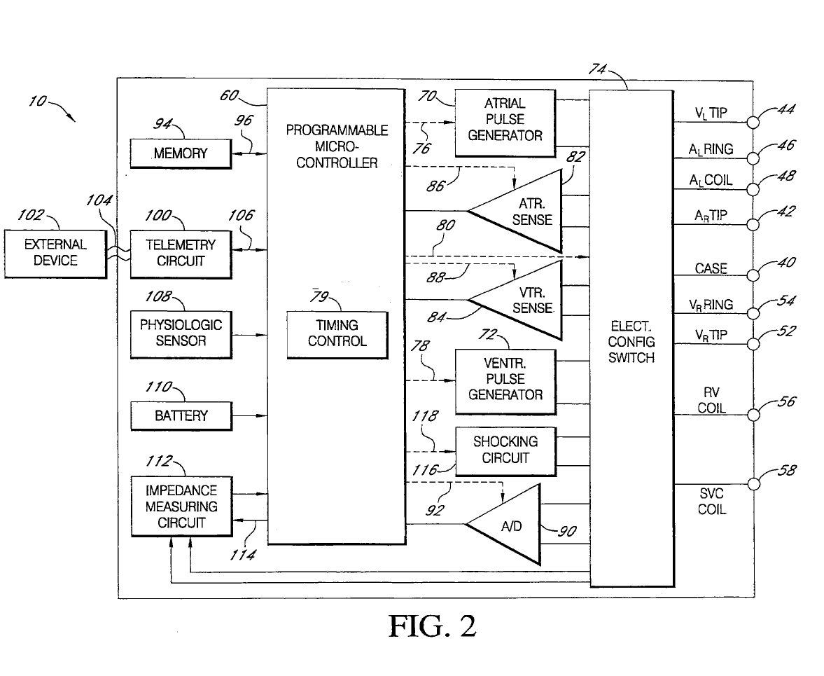

As illustrated in FIG. 2, a simplified block diagram is shown of the multi-chamber implantable stimulation device 10, which is capable of treating both fast and slow arrhythmias with cardioversion, defibrillation, and pacing stimulation. While a particular multi-chamber device is shown, this is for illustration purposes only and one of skill in the art could readily duplicate, eliminate or disable the appropriate circuitry in any desired combination to provide a device capable of treating the appropriate chamber(s) with cardioversion, defibrillation and pacing stimulation.

The housing 40 for the stimulation device 10, shown schematically in FIG. 2, is often referred to as the “can”, “case” or “case electrode” and may be programmably selected to act as the return electrode for all pacemaker “unipolar” modes. The housing 40 may further be used as a return electrode alone or in combination with one or more of the coil electrodes, 28, 36 and 38, for high voltage shocks. The housing 40 further includes a connector (not shown) having a plurality of terminals 42, 44, 46, 48, 52, 54, 56, and 58 (shown schematically and, for convenience, along with the names of the electrodes to which they are connected). As such, to achieve right atrial sensing and pacing, the connector includes at least a right atrial tip terminal (A_{R} TIP) 42 adapted for connection to the atrial tip electrode 22.

To achieve left chamber sensing, pacing and high voltage shocking, the connector includes at least a left ventricular tip terminal (V_{L} TIP) 44, a left atrial ring terminal (A_{L} RING) 46, and a left atrial shocking terminal (A_{L} COIL) 48, which are adapted for connection to the left ventricular tip electrode 26, the left atrial ring electrode 27, and the left atrial coil electrode 28, respectively.

To support right chamber sensing, pacing and high voltage shocking, the connector further includes a right ventricular tip terminal (V_{R} TIP) 52, a right ventricular ring terminal (V_{R} RING) 54, a right ventricular shocking terminal (R_{V} COIL) 56, and an SVC shocking terminal (SVC COIL) 58, which are adapted for connection to the right ventricular tip electrode 32, right ventricular ring electrode 34, the RV coil electrode 36, and the SVC coil electrode 38, respectively.

At the core of the stimulation device 10 is a programmable microcontroller 60 which controls the various modes of stimulation therapy. As is well known in the art, the microcontroller 60 typically includes a microprocessor, or equivalent control circuitry, designed specifically for controlling the delivery of stimulation therapy and may further include RAM or ROM memory, logic and timing circuitry, state machine circuitry, and I/O circuitry. Typically, the microcontroller 60 includes the ability to process or monitor input signals (data) as controlled by a program code stored in a designated block of memory. The details of the design and operation of the microcontroller 60 are not critical to the invention. Rather, any suitable microcontroller 60 may be used that carries out the functions described herein. The use of microprocessor-based control circuits for performing timing and data analysis functions are well known in the art.

As shown in FIG. 2, an atrial pulse generator 70 and a ventricular pulse generator 72 generate pacing stimulation pulses for delivery by the right atrial lead 20, the right ventricular lead 30, and/or the coronary sinus lead 24 via an electrode configuration switch 74. It is understood that in order to provide stimulation therapy in each of the four chambers of the heart, the atrial and ventricular pulse generators 70, 72 may include dedicated, independent pulse generators, multiplexed pulse generators, or shared pulse generators. The pulse generators 70, 72 are controlled by the microcontroller 60 via appropriate control signals 76, 78 respectively, to trigger or inhibit the stimulation pulses.

The microcontroller 60 further includes timing control circuitry 79 which is used to control the timing of such stimulation pulses (e.g., pacing rate, atrio-ventricular (AV) delay, atrial interconduction (A-A) delay, or ventricular interconduction (V-V) delay, etc.) as well as to keep track of the timing of refractory periods, noise detection windows, evoked response windows, alert intervals, marker channel timing, etc., which is well known in the art.

The switch 74 includes a plurality of switches for connecting the desired electrodes to the appropriate I/O circuits, thereby providing complete electrode programmability. Accordingly, the switch 74, in response to a control signal 80 from the microcontroller 60, determines the polarity of the stimulation pulses (e.g., unipolar, bipolar, combipolar, etc.) by selectively closing the appropriate combination of switches (not shown) as is known in the art. In this embodiment, the switch 74 also supports simultaneous high resolution impedance measurements, such as between the case or housing 40, the right atrial electrode 22, and right ventricular electrodes 32, 34 as described in greater detail below.

Atrial sensing circuits 82 and ventricular sensing circuits 84 may also be selectively coupled to the right atrial lead 20, coronary sinus lead 24, and the right ventricular lead 30, through the switch 74 for detecting the presence of cardiac activity in each of the four chambers of the heart. Accordingly, the atrial and ventricular sensing circuits 82, 84 may include dedicated sense amplifiers, multiplexed amplifiers, or shared amplifiers. The switch 74 determines the “sensing polarity” of the cardiac signal by selectively closing the appropriate switches, as is also known in the art. In this way, the clinician may program the sensing polarity independently of the stimulation polarity.

Each sensing circuit 82, 84 preferably employs one or more low power, precision amplifiers with programmable gain and/or automatic gain control, bandpass filtering, and a threshold detection circuit, as known in the art, to selectively sense the cardiac signal of interest. The automatic gain control enables the device 10 to deal effectively with the difficult problem of sensing the low amplitude signal characteristics of atrial or ventricular fibrillation. The outputs of the atrial and ventricular sensing circuits 82, 84 are connected to the microcontroller 60 which, in turn, are able to trigger or inhibit the atrial and ventricular pulse generators 70, 72 respectively, in a demand fashion in response to the absence or presence of cardiac activity in the appropriate chambers of the heart.

For arrhythmia detection, the device 10 utilizes the atrial and ventricular sensing circuits 82, 84 to sense cardiac signals to determine whether a rhythm is physiologic or pathologic. As used herein “sensing” is reserved for the noting of an electrical signal, and “detection” is the processing of these sensed signals and noting the presence of an arrhythmia. The timing intervals between sensed events (e.g., P-waves, R-waves, and depolarization signals associated with fibrillation) are then classified by the microcontroller 60 by comparing them to a predefined rate zone limit (i.e., bradycardia, normal, low rate VT, high rate VT, and fibrillation rate zones) and various other characteristics (e.g., sudden onset, stability, physiologic sensors, and morphology, etc.) in order to determine the type of remedial therapy that is needed (e.g., bradycardia pacing, anti-tachycardia pacing, cardioversion s or defibrillation with high voltage shocks, collectively referred to as “tiered therapy”).

Cardiac signals are also applied to the inputs of an analog-to-digital (A/D) data acquisition system 90. The data acquisition system 90 is configured to acquire intracardiac electrogram (IEGM) signals, convert the raw analog data into a digital signal, and store the digital signals for later processing and/or telemetric transmission to an external device 102. The data acquisition system 90 is coupled to the right atrial lead 20, the coronary sinus lead 24, and the right ventricular lead 30 through the switch 74 to sample cardiac signals across any pair of desired electrodes.

The microcontroller 60 is further coupled to a memory 94 by a suitable data/address bus 96, wherein the programmable operating parameters used by the microcontroller 60 are stored and modified, as required, in order to customize the operation of the stimulation device 10 to suit the needs of a particular patient. Such operating parameters define, for example, pacing pulse amplitude, pulse duration, electrode polarity, rate, sensitivity, automatic features, arrhythmia detection criteria, and the amplitude, waveshape and vector of each shocking pulse to be delivered to the patient's heart within each respective tier of therapy.

Advantageously, the operating parameters of the implantable device 10 may be non-invasively programmed into the memory 94 through a telemetry circuit 100 in telemetric communication with the external device 102, such as a programmer, transtelephonic transceiver, or a diagnostic system analyzer. The telemetry circuit 100 is activated by the microcontroller by a control signal 106. The telemetry circuit 100 advantageously allows IEGMs and status information relating to the operation of the device 10 (as contained in the microcontroller 60 or memory 94) to be sent to the external device 102 through an established communication link 104.

In the preferred embodiment, the stimulation device 10 further includes a physiologic sensor 108, commonly referred to as a “rate-responsive” sensor because it is typically used to adjust pacing stimulation rate according to the exercise state of the patient. However, the physiological sensor 108 may further be used to detect changes in cardiac output, changes in the physiological condition of the heart, or diurnal changes in activity (e.g., detecting sleep and wake states). Accordingly, the microcontroller 60 responds by adjusting the various pacing parameters (such as rate, AV Delay, V-V Delay, etc.) at which the atrial and ventricular pulse generators 70, 72 generate stimulation pulses.

The stimulation device 10 additionally includes a battery 110 which provides operating power to all of the circuits shown in FIG. 2. For the stimulation device 10, which employs high voltage shocking therapy, the battery 110 must be capable of operating at low current drains for long periods of time and then be capable of providing high-current pulses (for capacitor charging) when the patient requires a high voltage shock. The battery 110 must also have a predictable discharge characteristic so that elective replacement time can be detected. Accordingly, embodiments of the device 10 including high voltage shocking capability preferably employ lithium/silver vanadium oxide batteries. For embodiments of the device 10 not including high voltage therapy, the battery 110 will preferably be lithium iodide or carbon monoflouride or a hybrid of the two.

As further shown in FIG. 2, the device 10 is shown as having an impedance measuring circuit 112 which is enabled by the microcontroller 60 via a control signal 114.

In the case where the stimulation device 10 is intended to operate as an implantable cardioverter/defibrillator (ICD) device, it must detect the occurrence of an arrhythmia, and automatically apply appropriate high voltage shock therapy to the heart aimed at terminating the detected arrhythmia. To this end, the microcontroller 60 further controls a shocking circuit 116 by way of a control signal 118. The shocking circuit 116 generates shocking pulses of low (up to 0.5 joules), moderate (0.5-10 joules), or high energy (11 to 40 joules), as controlled by the microcontroller 60. The energy level of the shocking pulse is calculated by programming the amplitude and wave shape operating parameters of the pulse. Such shocking pulses are applied to the patient's heart 12 through at least two shocking electrodes, and as shown in this embodiment, selected from the left atrial coil electrode 28, the RV coil electrode 36, and/or the SVC coil electrode 38. As noted above, the housing 40 may act as an active electrode in combination with the RV electrode 36, or as part of a split electrical vector using the SVC coil electrode 38 or the left atrial coil electrode 28 (i.e., using the RV electrode as a common electrode).

Cardioversion shocks are generally considered to be of low to moderate energy level (so as to minimize pain felt by the patient), and/or synchronized with an R-wave and/or pertaining to the treatment of tachycardia. Defibrillation shocks are generally of moderate to high energy level (i.e., corresponding to thresholds in the range of 5-40 joules), delivered asynchronously (since R-waves may be too disorganized), and pertaining exclusively to the treatment of fibrillation. Accordingly, the microcontroller 60 is capable of controlling the synchronous or asynchronous delivery of the high voltage shocking pulses.

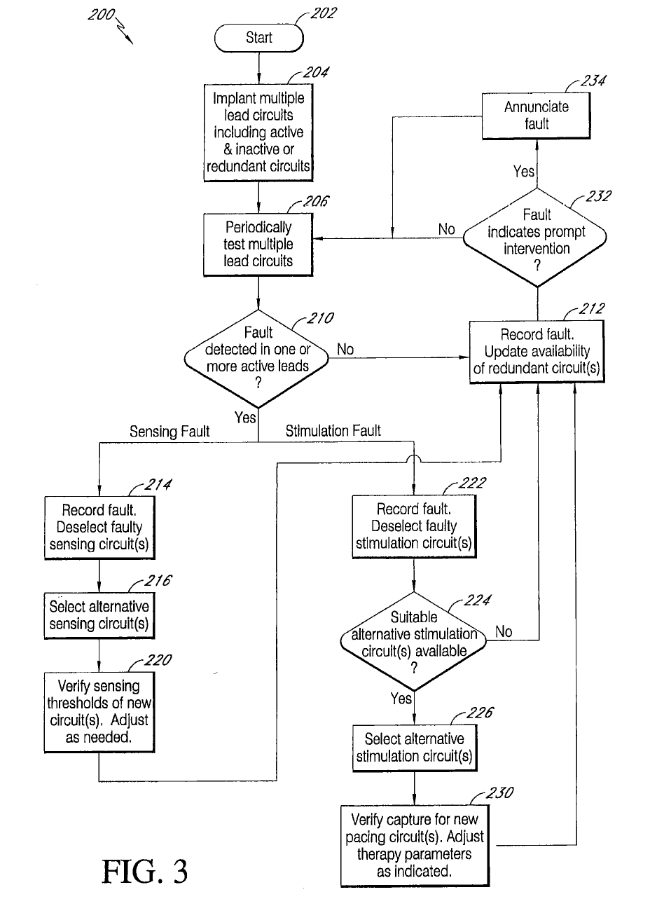

In FIG. 3, a flow chart is shown describing an overview of the operation and features implemented in embodiments of the device 10. In this flow chart, the various algorithmic steps are summarized in individual “blocks”. Such blocks describe specific actions or decisions that are made or carried out as the algorithm proceeds. Where a microcontroller (or equivalent) is employed, the flow charts presented herein provide the basis for a “control program” that may be used by such a microcontroller (or equivalent) to effectuate the desired control of the stimulation device 10. Those skilled in the art may readily write such a control program based on the flow charts and other descriptions presented herein.

FIG. 3 illustrates a flow chart of one embodiment of a system and method 200 for providing redundancy and safe mode operation of an implantable medical device. The system and method 200 begins in a start state 202. The start state 202 generally includes the previously described operation of the device for monitoring and indicated delivery of therapy to the patient. It will be understood that the additional steps, processes, and components of the system and method 200 would generally proceed in parallel with the other operations and processes of the device 10 as previously described.

The system and method 200 includes a state 204 wherein a plurality of electrodes are implanted in the patient and configured in one or more vector arrangements, i.e., lead circuits, for sensing and/or delivery of therapy to patient tissue. This plurality of lead circuits would include lead circuits which are designated or configured as active vectors or circuits, e.g., for the ongoing sensing and/or delivery of stimulation by the device 10 as previously described. The exact number, placement, and operating characteristics of the active lead circuits will depend both on the configuration of the device 10 itself, as well as the particular needs of a given patient. However, selection and adjustment of these factors will be well understood by one of ordinary skill considering the needs of the patient.

State 204 also includes configuring implanted electrodes in one or more inactive or redundant vector arrangement, i.e., lead circuits. The inactive or redundant lead circuits would otherwise be comparable to the active lead circuits, however, they are not initially selected or configured for sensing of physiologic activity or for delivery of therapeutic stimulation. The idle or redundant circuits are provided in state 204 to provide the availability of back-up lead circuits should one or more of the initially active lead circuits experience a problem. Again, selection of appropriate placement and available operating characteristics of the inactive or redundant circuits will be readily understood by one of ordinary skill.

In one embodiment, the inactive or redundant circuits are also initially evaluated for preliminary operating programming. In one exemplary embodiment, preliminary programming for sensing and/or delivery of stimulation, e.g., including determination of appropriate sensing amplifier gains and detection thresholds, stimulation capture thresholds, etc., is performed before the redundant leads are actually used. In certain embodiments, a preliminary evaluation is followed by a further evaluation should the redundant lead circuits be selected for active use to reverify their operation (e.g. blocks 220 and/or 230 below). In other embodiments, the evaluation and programming of the redundant lead circuits is only performed if the corresponding lead circuit is activated.

Thus, in state 204, a first group of lead circuits is programmed for active or enabled operation for providing the previously described monitoring of the patient's physiologic condition as well as indicated delivery of therapy. State 204 also provides a second set of lead circuits which are idled or disabled to provide redundancy or back-up capacity to the device 10. As one particular example, the device 10 can be configured in state 204 to include placement of electrodes and formation of one or more lead circuits in both the right ventricle and the left ventricle. These lead circuits can be further configured into a first set of lead circuits in an active state to provide sensing and indicated stimulation to one of the ventricles with the lead circuits in the other ventricle grouped as a second set of lead circuits in an inactive or idle state. Thus, in an initial configuration, the device 10 has the capability to provide sensing and stimulation delivery to opposed or paired heart chambers, however, it is initially configured to only actively sense and deliver therapy to one of the pair.

Following the start state 202 and state 204, is a test state 206 wherein the multiple lead circuits provided in state 204 are tested or evaluated for indications of problems in one or more of the leads carrying the electrodes that define the lead circuits. The problems tested or evaluated for in state 206 can include both lead conductivity problems arising from component damage, such as a partial or complete fracture or short in a lead conductor, as well as component/tissue interface problems, such as dislodgement of a lead electrode from an implanted location. A variety of lead integrity testing or evaluation systems and methodologies, for example, lead impedance testing, will be well understood by one of ordinary skill.

The testing of state 206 is in certain embodiments triggered by the device 10 following some indication of a possible lead circuit problem. Indications of possible lead circuit problems can constitute relatively abrupt changes in the operation or performance of the device 10. For example, the testing of state 206 can be initiated by the triggering of an excessive number or frequency of autocapture threshold searches initiated by the device 10. In many embodiments, the device 10 automatically periodically performs autocapture threshold searches wherein the pulse energy of stimulation waveforms required to capture the activity of the heart 12 with stimulation is automatically determined. In one embodiment, a sharp increase in the frequency and/or number of these autocapture threshold searches initiated by the device 10 can indicate that some lead circuit problem has occurred such that the efficacy of the existing stimulation as delivered to the target tissue is degraded, thereby limiting the ability of the device 10 to effect capture with the existing stimulation parameters. Autocapture can be repeated to determine the pulse energy required for capture, however a sharp increase in the frequency/number of times with which this occurs may indicate that the change is due to some lead circuit problem.

In another embodiment, the testing of state 206 is triggered by the onset of increased incidences of under- or over-sensing indications, which may be indicative of a fault with a lead circuit sensing function. Undersensing occurs when the device 10 fails to properly detect cardiac activity which is occurring. Oversensing occurs when the device 10 inappropriately overcounts the detection of cardiac activity actually occurring, for example by inaccurately detecting multiple occurrences of an R-wave within a single cardiac cycle. Undersensing can result in an inappropriate interpretation of a bradycardia condition and oversensing can likewise result in inaccurate observation of tachycardia. Thus, in certain embodiments, relatively abrupt onset and/or termination of bradycardia/tachycardia indications, arrhythmia indications that are not corroborated by other physiologic indicators, and/or such indications that are inconsistent with known histories of such activity in the patient may indicate possible lead circuit problems that trigger the testing of state 206.

The testing of state 206 can be triggered in yet a further embodiment by a marked change in a capture threshold indicative of a problem with delivery of stimulation via one or more lead circuits. In yet another embodiment, testing in state 206 can be triggered by an out of range condition exhibited by a routine lead impedance test. Thus, in various embodiments, the testing of state 206 can be triggered asynchronously by the passive observation of any condition or indication that a lead circuit problem may limit or prevent the ability of the device 10 to properly sense and/or deliver therapy via the affected lead circuit(s). The observation for possible lead circuit problems can be performed by the device 10, in certain embodiments, as a passive adjunct to other normal ongoing operation of the device 10 and can piggyback onto or employ other processes that are not dedicated solely to lead testing. Thus, the testing of state 206 can be triggered in certain embodiments by any of a number of anomalous conditions and is not limited strictly to indications of component damage.

In yet other embodiments which can be provided as an alternative or in combination with the triggered or a periodic testing of state 206 previously described, the testing can also occur on a regular periodic basis. For example, testing of state 206 can be programmed to occur on a daily basis or other regular interval as selected by the clinician. In further embodiments, the testing of state 206 can occur on a regular periodic basis, however, wherein the interval or period is adjustable or a function of another characteristic. For example, the system and method 200 can automatically monitor battery capacity and as battery capacity decreases, can adjust the interval between successive incidence of the testing of state 206, for example, to extend the period to thereby reduce drain on the battery or to shorten the interval or period to more frequently confirm the integrity of the lead circuits with reduced battery capacity.

Should the testing of state 206 return indications of problems or fault in one or more of the lead circuits, a decision or evaluation state 210 follows. In the decision state 210, a determination is made as to whether the fault detected in the testing of state 206 is in one or more of the active or first lead circuits. If the evaluation of state 210 is negative, the system and method 200 proceeds to a state 212 wherein the fault in a non-active lead circuit is recorded. The state 212 also includes updating of an availability or status record of the multiple lead circuits, and in one embodiment, this includes updating the status of both the active or first lead circuits, the redundant or idle second lead circuits, as well as the problem or fault status of any of the lead circuits.

Following from the recording and updating of state 212 is a further decision state 232 wherein an evaluation or decision is made as to whether the recently detected faults indicate prompt clinical intervention, such as replacement or repair of the one or more leads carrying the electrodes that define the affected lead circuits. For example, in certain implementations, a fault or a problem may occur in a redundant or idle circuit which, while providing a valuable useful redundancy, does not constitute a critical back-up or redundancy. However, in other implementations, a lead circuit fault does constitute a critical issue indicating prompt attention to address the fault. Thus, if the decision or evaluation of state 232 is negative and prompt intervention is not necessary, the system and method returns to normal ongoing operation awaiting the next incidence of the testing state 206. However, if the evaluation of state 232 is affirmative, e.g., that prompt intervention is indicated, a state 234 follows wherein an annunciation is provided to notify the patient that prompt attention is indicated. The annunciation of state 234 can comprise generation of an audible tone, generation of a tactile muscular stimulation, for example, stimulation of the pectoral muscles adjacent an implant location, and/or generation of a telemetric warning to the external device 102 to communicate the problem detection.

If the decision or evaluation of state 210 is affirmative, e.g., that a lead circuit fault or problem has been detected in one or more of the first or active lead circuits, the system and method 200 provide a variety of automated options or interventions to provide a safe mode type of operation. More particularly, options and actions provided by the system and method 200 can vary or branch depending upon whether individual lead circuit faults detected are sensing faults or stimulation faults. For example, as previously described, lead circuit faults can occur which are of a partial or progressive nature and thus a lead circuit can exhibit, for example, a sensing fault or limitation but still maintain adequate performance for delivery of therapeutic stimulation.

For sensing faults, the system and method 200 includes a state 214 wherein the fault is recorded. In one embodiment, the faulty sensing lead circuit(s) is/are also deselected for further use. Then, in a state 216, alternate sensing circuit(s) is selected. The selection of an alternative sensing lead circuit in state 216 in one embodiment comprises the selection of a different combination of electrodes associated with a particular implantable lead, such as among the leads 20, 24, 30. In one exemplary implantation, the device 10 can be initially configured or programmed to perform sensing between a tip and a ring electrode. However, if a fault occurs in the lead conductor connected to the tip electrode, the device 10 can revert or select an alternative lead circuit defined by a different combination of electrodes, for example, to sense between a ring and a coil electrode of the affected lead. In another exemplary implementation, rather than sensing between the ring electrode and a problematic tip electrode, the device 10 can revert or select to sense instead between the intact ring electrode and the case or housing 40 of the device 10. In yet another exemplary implantation, the device 10 can automatically select a lead sensing circuit positioned in an adjacent cardiac chamber. For example, the device 10 can be configured initially to sense activity in the left ventricle alone, however, in state 204 it is provided with redundant or back-up second sensing circuits in the right ventricle. Upon compromise of the sensing circuit to the left ventricle, the device 10 can select to sense instead from the adjacent right ventricle.

This aspect of the system and method 200 provides a valuable redundancy or safe mode of operation, as certain configurations of patient leads are at least partially of a unipolar or single conductor configuration. For example, in certain applications, patient leads may only include a single electrode and associated insulated conductor for placement, for example, in the left ventricle. Should such a single conductor electrode arrangement experience a problem, there can be no available further immediately adjacent electrodes, such as a ring electrode present in the immediate vicinity of the problematic electrode. However, by providing the ability to select an alternative sensing lead circuit, for example, a lead circuit defined by electrodes arranged in the adjacent right ventricle, the device 10 can continue to sense ventricular activity, even if under revised conditions.

Following the selection of alternative sensing circuits in state 216, the system and method 200 includes a reverification and adjustment state 220. As the particular configuration of sensing lead circuits has been revised in state 216, it will be expected that in certain implementations, the sensed signals observed with the newly selected lead circuits will vary at least somewhat from the previously active sensing lead circuits prior to the fault or problem. Thus, in state 220, the device 10 automatically reevaluates the sensing performance with the newly selected sensing circuits, for example, by performing a sensing threshold test. State 220 also includes adjusting the operating characteristics or parameters of the device 10 with the newly selected sensing circuits as indicated. It will be expected that in certain implementations, such as selection of an alternative combination of electrodes which are otherwise adjacent in location and similar in performance to the problematic electrodes, the indicated adjustment and subsequent sensing performance with the newly selected circuits will be comparable to the previous performance of the prefault initial sensing circuits. In other implementations, for example, where the newly selected alternative sensing circuit is in a substantially different location of the patient's heart, of substantially different size or performance, etc., the adjustment performed in state 220 may not result in alternative sensing circuit performance which is fully comparable to the prefault performance. However, the selection and adjustment of alternative circuits provided by the system and method 200 provides a valuable back-up or safe mode of operation to what otherwise might be a significant degradation in the ability of the device 10 to properly sense the patient's cardiac activity.

In cases of stimulation lead circuit faults, the system and method 200 includes a state 222 wherein the fault is recorded and the faulty stimulation circuits are deselected. Then follows a state 224 wherein an evaluation or determination is made as to whether suitable alternative stimulation lead circuits are available in the idle or redundant second group of lead circuits. As the therapeutic stimulation is targeted to specific regions of patient tissue, in certain implementations, a suitable alternative lead circuit may not exist to appropriately deliver certain types of stimulation in case of degradation of the respective stimulation lead circuits. Thus, if the determination of state 224 is negative and no suitable alternative circuits are available, the system and method proceeds to the state 212 and subsequent evaluation of state 232 whether the fault is serious enough to indicate prompt intervention.

If however, the determination of state 224 is affirmative, e.g., that a suitable alternative stimulation lead circuit is available, the system and method 200 proceeds to a state 226 wherein the appropriate alternative stimulation lead circuit or circuits are selected. Then, in a state 230, the newly selected alternative stimulation lead circuits are evaluated and adjusted as indicated. In one particular embodiment, this can include a reverification of capture for the new stimulation circuits. In an analogous manner to that previously described with respect to the verification and adjustment of state 220 for sensing lead circuits, it will be expected that in certain embodiments, any adjustment for newly selected stimulation circuits of state 230 can be of a relatively minor nature for new lead circuits which are similar in location and performance to the problematic stimulation lead circuit. In other embodiments, however, the required adjustment for improved stimulation performance can be significant and the resulting performance with the alternative stimulation lead circuits may not be fully comparable to the prefault initial stimulation lead circuits. Again, however, the system and method 200 provides valuable back-up or safe mode stimulation therapy availability.

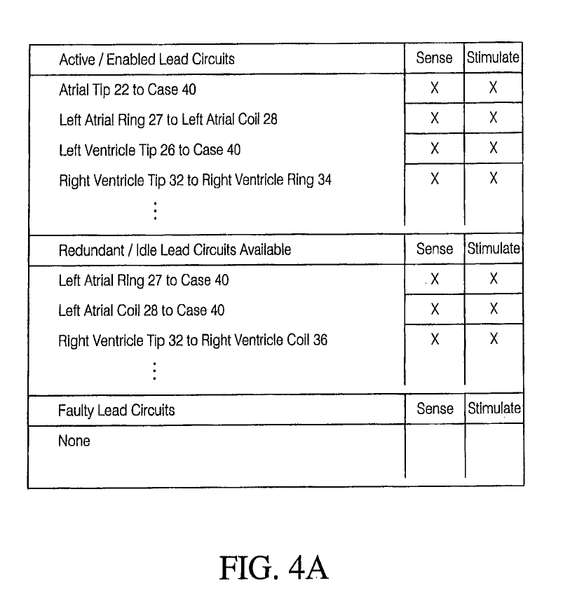

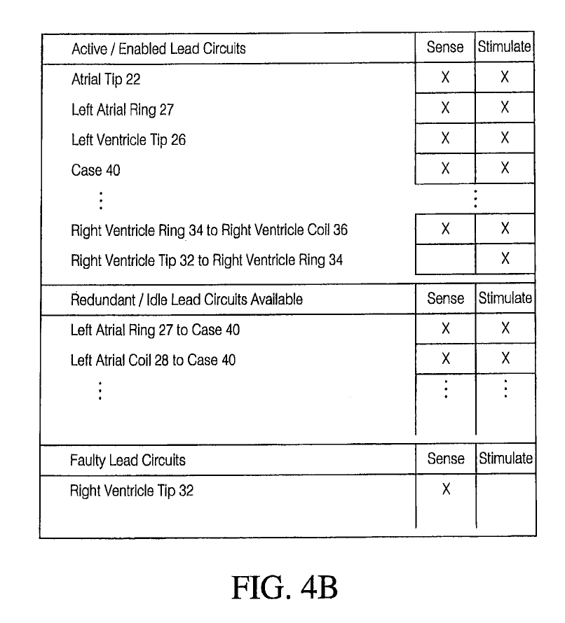

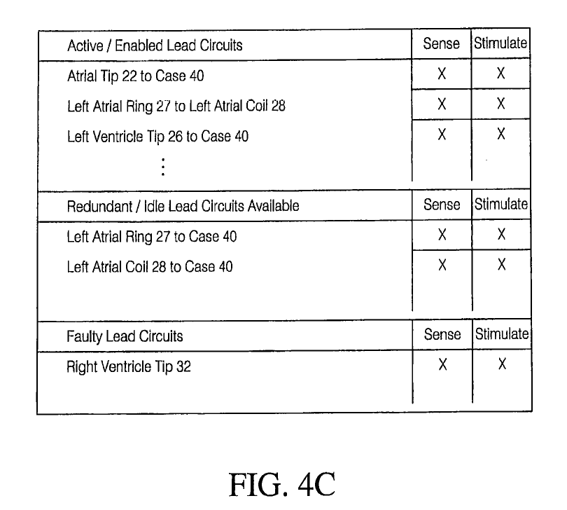

FIGS. 4A, 4B, and 4C illustrate various exemplary embodiments of a status record of the plurality of lead circuits of the device 10. The status record is periodically updated and recorded as previously described in the system and method 200, and the status record would generally be stored in the memory 94 of the device 10. FIG. 4A illustrates an initial or prefault status record indicating a number of active or enabled first lead circuits. The status record indicates, as illustrated with checks, the active or enabled state, both for sensing and stimulation. It will be understood that the number of individual electrodes of the device, as well as the resulting combinations in which they can be utilized, can be substantial, and the illustrations of FIGS. 4A, 4B, 4C are for illustrative purposes only and are not to be interpreted as exhaustive. It will also be understood that the particular combinations of electrodes utilized to define active lead circuits will vary depending upon the particular indications of a patient's needs and that these needs may change over time due to, for example, progression of their disease and/or revision of therapy regimens as determined by a clinician.

FIG. 4A also illustrates a number of idle or redundant second lead circuits which are available and similarly indicate their state both for sensing and stimulation. For example, FIG. 4A illustrates that a lead circuit is available between the left atrial ring electrode 27 and the case electrode 40, both for sensing and stimulation, however, this lead circuit is currently idle constituting a redundant but available lead circuit. FIG. 4A also illustrates record fields for detected faulty lead circuits in both their sensing and stimulation capacity. However, in this embodiment, none of the lead circuits, either of the active first lead circuits or idle second lead circuits, have been determined to be faulty for either of sensing or stimulation.

FIG. 4B illustrates another embodiment of a status record in this embodiment following at least one determination of a faulty lead circuit. More particularly, FIG. 4B illustrates that, for example, the atrial tip electrode 22, the left atrial ring electrode 27, the left ventricle tip electrode 26, and a number of other lead circuits are still intact for both sensing and stimulation. FIG. 4B however also indicates that the lead circuit of the right ventricle tip electrode 32 to the right ventricle ring electrode 34 is active and intact for stimulation purposes, however, is not active or enabled for sensing purposes. FIG. 4B also illustrates that this embodiment of a status record indicates a sensing fault for the right ventricle tip electrode 32. Again, the faults or problems addressed by the system and method 200 can involve both component damage, such as a fracture in a conductive element associated with a given electrode, and can also include component tissue interface faults, such as dislodgment of an otherwise intact electrode from its desired position and contact with target patient tissue.

FIG. 4C illustrates yet a further embodiment of a status record indicating in this embodiment a problem of the right ventricle tip electrode 32 for both sensing and stimulation. Again, such a problem can correspond to component damage, such as a lead fracture, as well as an interface problem, such as a dislodgement. In this embodiment, the right ventricle tip electrode 32 would be deselected or deactivated for both sensing and stimulation and would also not be available as an idle or redundant lead circuit, and thus would be removed from both the first and the second lead circuit groups, residing instead in a third or faulty lead circuit group.

Thus, the system and method 200 provides the ability to operate the device 10 in a safe mode that automatically accommodates and reverts for at least certain lead problems. The system and method 200 provides redundant or back-up lead circuits which can be employed as needed should initial or primary active lead circuits experience problems or damage. The system and method 200 provides the ability to discriminate between sensing and stimulation lead circuit faults, and provides the ability to continue to provide therapy delivery via a given lead circuit, even if the ability of that same lead circuit is compromised with respect to sensing performance.

The system and method 200 also provides the ability to provide a back-up or safe mode of operation for lead circuits which are unipolar or single conductor in nature. For example, certain lead configurations, such as those extending into the left ventricle, can have only a single associated electrode and lead conductor. Should such a single conductor lead suffer damage, the system and method 200 provides the ability to continue to sense and provide stimulation in the right ventricle, even if at somewhat reduced performance. The system and method 200 also provide the ability not only to provide redundant or back-up lead circuits, but to periodically verify their availability should lead damage occur. The system and method 200 further provide the capability that should such a redundant or back-up lead circuit or an actively employed lead circuit experience a problem of significant implication, the device 10 can provide an annunciation to alert the patient and/or attending clinical personnel of the problematic occurrence. The system and method 200 also provide the advantage and ability to evaluate and adjust the operating performance of redundant or back-up circuits should they be called into service as active lead circuits.

Although the above disclosed embodiments of the present teachings have shown, described and pointed out the fundamental novel features of the invention as applied to the above-disclosed embodiments, it should be understood that various omissions, substitutions, and changes in the form of the detail of the devices, systems and/or methods illustrated may be made by those skilled in the art without departing from the scope of the present teachings. Consequently, the scope of the invention should not be limited to the foregoing description but should be defined by the appended claims.

What is claimed is:

1. A method of monitoring a patient and providing therapy via an implantable medical device, said method comprising:
implanting a plurality of electrodes in, on or near the patient's heart;
initially configuring the plurality of electrodes to define first circuits enabled for at least one of sensing and stimulating and second circuits which are idle for at least one of sensing and stimulating;
testing selected first circuits or second circuits for fault indications related to one or both of sensing and stimulating;
updating a status record to indicate corresponding sensing fault indications and stimulating fault indications;
if a sensing fault is found in one of the first circuits, redefining the first circuit when enabled for sensing to include at least one electrode of a second circuit that does not have a record of a sensing fault indication; and
if a stimulating fault is found in one of the first circuits, redefining the first circuit when enabled for stimulating to include at least one electrode of a second circuit that does not have a record of a stimulating fault indication.

2. The method of claim 1 wherein at least one defined first circuit and one defined second circuit are arranged with respect to a common heart chamber.

3. The method of claim 1 wherein at least one defined first circuit and one defined second circuit are arranged with respect to different heart chambers.

4. The method of claim 3 wherein the different heart chambers comprise the left ventricle and the right ventricle.

5. The method of claim 1 wherein the testing is triggered by an observed anomaly in one of the first circuits.

6. The method of claim 5 wherein the observed anomaly comprises an excessive rate or number of initiated automatic capture threshold searches in a first circuit when it is enabled for stimulating.

7. The method of claim 5 wherein the observed anomaly comprises an increase in at least one of over-sensing and under-sensing indications in a first circuit when it is enabled for sensing.

8. The method of claim 1 wherein the testing is performed on a regular periodic basis.

9. The method of claim 1 wherein redefining the first circuit when enabled for sensing, comprises retaining the initial electrode configuration of the first circuit when enabled for stimulating.

10. The method of claim 1 wherein redefining the first circuit when enabled for stimulating, comprises retaining the initial electrode configuration of the first circuit when enabled for sensing.

11. The method of claim 1, further comprising, following redefining of one of the first circuits, testing the redefined first circuit and adjusting operating parameters of the device with the revised first circuit.

12. An implantable cardiac stimulation device comprising:
an implantable stimulation generator;
a plurality of implantable electrodes; and
a controller in communication with the stimulation generator and the plurality of electrodes wherein the controller:
activates selected ones of the plurality of electrodes to form an active circuit for at least one of sensing physiologic activity and delivering therapy from the stimulation generator to patient tissue;
idles selected other ones of the plurality of electrodes to form one or more idle circuits as alternatives to the active circuit for possible later activation;
evaluates the active circuit for problem indications and, upon detecting a problem, designates the active circuit as unavailable and activates at least one of the idled circuits; and
memory in communication with the controller and configured to store a status record of the active, idle, or unavailable state of the plurality of circuits.

13. The device of claim 12 wherein the active circuit and one of its corresponding idle circuits are arranged with respect to the same heart chamber.

14. The device of claim 12 wherein the active circuit and one of its corresponding idle circuits are arranged with respect to adjacent heart chambers.

15. The device of claim 14 wherein the adjacent heart chambers comprise the left ventricle and the right ventricle.

16. The device of claim 12 wherein when the active circuit is activated for both sensing and delivering therapy, the controller:
evaluates the active circuit for sensing problems;
upon detecting a sensing problem, designates the active circuit as unavailable for sensing but still available for delivering therapy; and
activates an idle circuit for sensing purposes only.

17. The device of claim 12 wherein when the active circuit is activated for both sensing and delivering therapy, the controller:
evaluates the active circuit for therapy delivery problems;
upon detecting a therapy delivery problem, designates the active circuit as unavailable for therapy delivery but still available for sensing; and
activates an idle circuit for therapy delivery purposes only.

18. The device of claim 12 wherein the active circuit and at least one of its idle circuits share a common electrode.
